(19) United States
(12) Patent Application Publication
(10) Pub. No.: US 2024/0165104 A1
(43) Pub. Date: May 23, 2024

(54) TREATMENT OF DEPRESSION

(71) Applicant: ANTECIP BIOVENTURES II LLC, New York, NY (US)

(72) Inventor: Herriot Tabuteau, New York, NY (US)

(21) Appl. No.: 18/418,010

(22) Filed: Jan. 19, 2024

Related U.S. Application Data
(63) Continuation of application No. PCT/US2022/037913, filed on Jul. 21, 2022.
(60) Provisional application No. 63/224,323, filed on Jul. 21, 2021.

(51) Int. Cl. A61K 31/485; A61K 31/137; A61P 25/24

(52) U.S. Cl. CPC A61K 31/485; A61K 31/137; A61P 25/24

(57) ABSTRACT

This disclosure relates to administration of a combination of: 1) about 100-110 mg, about 104-106 mg, or about 105 mg of bupropion hydrochloride, or a molar equivalent amount of a free base form or another salt form of dextromethorphan; and 2) about 40-50 mg, about 44-46 mg, or about 45 mg of dextromethorphan hydrobromide, or a molar equivalent amount of a free base form or another salt form of dextromethorphan for treating depression in patients with certain levels of dextromethorphan.

### CROSS REFERENCE TO RELATED APPLICATIONS

This application is a continuation of PCT Pat. App. No. PCT/US2022/037913, filed Jul. 21, 2022; which claims the benefit of U.S. Prov. Pat. App. No. 63/224,323, filed Jul. 21, 2021; all of which are incorporated by reference in their entirety.

### SUMMARY

This disclosure relates to administration of a combination of: 1) about 100-110 mg, about 104-106 mg, or about 105 mg of bupropion hydrochloride, or a molar equivalent amount of a free base form or another salt form of bupropion; and 2) about 40-50 mg, about 44-46 mg, or about 45 mg of dextromethorphan hydrobromide, or a molar equivalent amount of a free base form or another salt form of dextromethorphan in certain patient populations.

Some embodiments include a method of treating major depressive disorder, comprising administering a combination comprising 45 mg of dextromethorphan hydrobromide, or a molar equivalent amount of another salt form or the free base form of dextromethorphan, and 105 mg of bupropion hydrochloride, or a molar equivalent amount of another salt form or the free base form of bupropion, twice a day for at least six weeks to a human patient in need thereof, wherein, twelve hours after a dose of the combination is administered on day 42, the human patient has a plasma level of dextromethorphan that is at least about 19 ng/mL.

Some embodiments include a method of treating major depressive disorder, comprising administering a dosage form comprising 45 mg of dextromethorphan hydrobromide, or a molar equivalent amount of another salt form or the free base form of dextromethorphan, and 105 mg of bupropion hydrochloride, or a molar equivalent amount of another salt form or the free base form of bupropion, twice a day for at least six weeks to a human patient in need thereof, wherein twenty-four hours after administration of the dosage form is discontinued, the human patient has a plasma level of dextromethorphan that is at least about 15 ng/mL.

Some embodiments include a method of treating major depressive disorder, comprising administering a dosage form comprising 45 mg of dextromethorphan hydrobromide, or a molar equivalent amount of another salt form or the free base form of dextromethorphan, and 105 mg of bupropion hydrochloride, or a molar equivalent amount of another salt form or the free base form of bupropion, twice a day for at least six weeks to a human patient in need thereof, wherein at any time after the eighth day of administering the combination, the human patient has a plasma level of dextromethorphan that is at least about 15 ng/mL.

Some embodiments include a drug combination for treating major depressive disorder, comprising 45 mg of dextromethorphan hydrobromide, or a molar equivalent amount of another salt form or the free base form of dextromethorphan, and 105 mg of bupropion hydrochloride, or a molar equivalent amount of another salt form or the free base form of bupropion, wherein the drug combination is administered twice a day for at least six weeks to a human patient in need thereof, and wherein, twelve hours after a dose of the combination is administered on day 42, the human patient has a plasma level of dextromethorphan that is at least about 19 ng/mL.

Some embodiments include a dosage form for treating major depressive disorder, comprising 45 mg of dextromethorphan hydrobromide, or a molar equivalent amount of another salt form or the free base form of dextromethorphan, and 105 mg of bupropion hydrochloride, or a molar equivalent amount of another salt form or the free base form of bupropion, wherein the dosage form is administered twice a day for at least six weeks to a human patient, and wherein twenty-four hours after administration of the dosage form is discontinued, the human patient has a plasma level of dextromethorphan that is at least about 15 ng/mL.

Some embodiments include use of a combination of dextromethorphan and bupropion in the manufacture of a medicament for treating major depressive disorder, wherein the combination comprising 45 mg of dextromethorphan hydrobromide, or a molar equivalent amount of another salt form or the free base form of dextromethorphan, and 105 mg of bupropion hydrochloride, or a molar equivalent amount of another salt form or the free base form of bupropion, is administered twice a day for at least six weeks to a human patient in need thereof, wherein, twelve hours after a dose of the combination is administered on day 42, the human patient has a plasma level of dextromethorphan that is at least about 19 ng/mL.

Some embodiments include use of a combination of dextromethorphan and bupropion in the manufacture of a medicament for treating major depressive disorder, wherein the combination comprising 45 mg of dextromethorphan hydrobromide, or a molar equivalent amount of another salt form or the free base form of dextromethorphan, and 105 mg of bupropion hydrochloride, or a molar equivalent amount of another salt form or the free base form of bupropion, is administered twice a day for at least six weeks, wherein twenty-four hours after administration of the medicament is discontinued, the patient has a plasma level of dextromethorphan that is at least about 15 ng/mL.

### BRIEF DESCRIPTION OF THE DRAWINGS

FIG. 1 is a plot of the MADRS total score change from baseline over time for the subjects described in Example 1.

FIG. 2 is a plot of the QIDS-SR-16 total score change from baseline over time for the subjects described in Example 1.

FIG. 3 is a plot of the percentage of subjects in remission (QIDS-SR-16 Score≤5) over time for the subjects described in Example 1.

FIG. 4 is a plot of the Cognitive Items of MGH-CPFQ change from baseline over time for the subjects described in Example 1.

FIG. 5 is a plot of the MADRS total score change from baseline over time for the subjects described in Example 2.

FIG. 6 is a plot of the Sheehan Disability Scale (SDS) mean domain score change from baseline over time for the subjects described in Example 2.

FIG. 7A is a chart of the percentage of subjects having clinical response based upon MADRS improvement (with MADRS improvement ≥50%) as compared to bupropion alone for the subjects described in Example 2.

FIG. 7B is a chart of the percentage of subjects having clinical remission (with MADRS≤10) based upon MADRS improvement as compared to bupropion alone for the subjects described in Example 2.

FIG. 8 is a plot of the changes in HAM-A and MADRS inner tension item from baseline over time for the subjects described in Example 2.

FIG. 9 is a plot of the changes in CPFQ-Cognition and MADRS concentration scores from baseline over time for the subjects described in Example 2.

### DETAILED DESCRIPTION

As mentioned above, this disclosure relates to administration of a combination of: 1) about 100-110 mg, about 104-106 mg, or about 105 mg of bupropion hydrochloride, or a molar equivalent amount of a free base form (such as 91 mg of bupropion free base) or another salt form of dextromethorphan; and 2) about 40-50 mg, about 44-46 mg, or about 45 mg of dextromethorphan hydrobromide, or a molar equivalent amount of a free base form (such as 33 mg of dextromethorphan free base) or another salt form of dextromethorphan. This combination is referred to for convenience herein as the “subject combination.” In every instance where the subject combination is referred to herein, the combination of 105 mg of bupropion hydrochloride (or 91 mg of bupropion free base) and 45 mg of dextromethorphan hydrobromide (or 33 mg of dextromethorphan free base) is specifically contemplated.

Dextromethorphan hydrobromide is an uncompetitive NMDA receptor antagonist and A sigma-1 receptor agonist.

It has been discovered that measurement of plasma drug concentrations is a direct method of assessing treatment adherence for the use of the combination of dextromethorphan and bupropion. Treatment adherence may improve the outcome with the use of the combination of dextromethorphan and bupropion since the attainment of therapeutic concentrations may be predicated not only on dose and frequency of the study drug but also on the time-dependent inhibition of CYP2D6.

Treatment adherence for a combination of 45 mg of dextromethorphan hydrobromide (or a molar equivalent amount of another salt form or a free base form) and 105 mg of bupropion hydrochloride (or a molar equivalent amount of another salt form or the free base form) may be determined based upon a dextromethorphan plasma concentration of at least 15 ng/mL at any time after about eight days of treatment, including at 24 hours after the last dose of the combination is administered. This also corresponds to a dextromethorphan plasma concentration of at least about 19 ng/mL at 12 hours after a dose administered once the levels of the drugs have reach steady state, such as at about 8 days, at about 14 days, at about 28 days, at about 42 days, etc.

In some embodiments, a blood sample is taken of a patient at any time after eight days of administering the drug combination. If the sample shows that the patient had a plasma concentration of dextromethorphan that was less than 15 ng/mL, the patient is encouraged to be more compliant in taking the drug combination and/or the treatment is discontinued in favor of a treatment with which the patient will be compliant.

The chemical name of dextromethorphan hydrobromide is morphinan, 3-methoxy-17-methyl-, (9α, 13α, 14α), hydrobromide monohydrate. Dextromethorphan hydrobromide has the empirical formula C₁₈H₂₅NO·HBr·H₂O and a molecular weight of 370.33. The structural formula is:

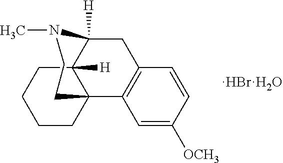 [SMILES: COc1ccc2c(c1)[C@]13CCCC[C@@H]1[C@H](C2)N(C)CC3]

Dextromethorphan hydrobromide powder is white or almost white, crystalline, and sparingly soluble in water.

Bupropion hydrochloride is an aminoketone and CYP450 2D6 inhibitor.

The chemical name of bupropion hydrochloride is: (±)-1-(3-chlorophenyl)-2-[(1,1-dimethylethyl)amino]-1-propanone hydrochloride. Bupropion hydrochloride has the empirical formula C₁₃H₁₈CINO·HCl and a molecular weight of 276.2. The structural formula is:

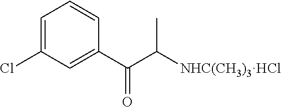

Bupropion hydrochloride powder is white and highly soluble in water.

The subject combination may be contained in an oral dosage form, including a tablet, such as an extended-release tablet. In some embodiments, the subject combination is contained in a dosage form for oral administration and is available as round bilayer tablets.

In some embodiments, each tablet containing the subject combination contains 45 mg of dextromethorphan hydrobromide in an immediate-release formulation. In some embodiments, each tablet of the subject combination contains 105 mg of bupropion hydrochloride in an extended-release formulation. In some embodiments, each tablet of the subject combination contains 45 mg of dextromethorphan hydrobromide in an immediate-release formulation and 105 mg of bupropion hydrochloride in an extended-release formulation.

In some embodiments, a tablet containing the subject combination contains I-cysteine hydrochloride monohydrate. In some embodiments, a tablet containing the subject combination contains carbomer homopolymer. In some embodiments, a tablet containing the subject combination contains microcrystalline cellulose. In some embodiments, a tablet containing the subject combination contains colloidal silicon dioxide. In some embodiments, a tablet containing the subject combination contains crospovidone. In some embodiments, a tablet containing the subject combination contains stearic acid. In some embodiments, a tablet containing the subject combination contains magnesium stearate.

In some embodiments, a tablet containing the subject combination contains the following inactive ingredients: I-cysteine hydrochloride monohydrate, carbomer homopolymer, microcrystalline cellulose, colloidal silicon dioxide, crospovidone, stearic acid, and magnesium stearate.

In some embodiments, the starting dosage of the subject combination is 45 mg of dextromethorphan hydrobromide and 105 mg of bupropion hydrochloride in one tablet that is administered once daily in the morning. In some embodiments, after 3 days, the dosage is increased to one tablet (or one dosage form containing 45 mg of dextromethorphan hydrobromide and 105 mg of bupropion hydrochloride) twice daily, e.g., given at least 8 hours apart. In some embodiments, no more than two doses containing 45 mg of dextromethorphan hydrobromide and 105 mg of bupropion hydrochloride are administered in the same day.

The subject combination may be administered orally with or without food. In some embodiments, the tablets are swallowed whole, and not crushed, divided, or chewed.

Steady state plasma concentrations of dextromethorphan and bupropion when given as the subject combination are achieved within 8 days. The accumulation ratios for dextromethorphan at steady state are about 20 and about 32, respectively based on Cₘₐₓ and AUC₀₋₁₂. The accumulation ratios for bupropion at steady state are 1.1 and 1.5, respectively based on Cₘₐₓ and AUC₀₋₁₂.

After administration of the subject combination, the median Tₘₐₓ of dextromethorphan is about 3 hours and the median Tₘₐₓ of bupropion is about 2 hours. The Cₘₐₓ of hydroxybupropion metabolite occurs approximately 3 hours post-dose and is approximately 14 times the peak level of bupropion. The AUC₀₋₁₂ hydroxybupropion is about 19 times that of bupropion. The Cₘₐₓ of the erythrohydroxybupropion and threohydroxybupropion metabolites occurs approximately 4 hours post-dose and is approximately equal to and about 5 times that of bupropion, respectively. The AUC₀₋₁₂ values of erythrohydroxybupropion and threohydroxybupropion are about 1.2 and about 7 times that of bupropion, respectively.

The subject combination can be taken with or without food. Dextromethorphan Cₘₐₓ and AUC₀₋₁₂ were unchanged and decreased by 14%, respectively, and bupropion Cₘₐₓ and AUC₀₋₁₂ were increased by 3% and 6%, respectively, when the subject combination was administered with food.

The plasma protein binding of dextromethorphan is approximately 60-70% and bupropion is 84%. The extent of protein binding of the hydroxybupropion metabolite is similar to that for bupropion; whereas the extent of protein binding of the threohydroxybupropion metabolite is about half that seen with bupropion.

Following 8 days of administration of the subject combination in extensive metabolizers, the mean elimination half-life of dextromethorphan was increased approximately 3-fold to about 22 hours, as compared to dextromethorphan given without bupropion.

The mean elimination half-life of dextromethorphan and bupropion was 22 hours and 15 hours, respectively. The apparent elimination half-life of hydroxybupropion, erythrohydroxybupropion and threohydroxybupropion metabolites were approximately 35, 44 and 33 hours, respectively.

In addition to major depressive disorder, the subject combination may be used to treat other diseases in conditions in the patient populations or circumstances described herein. For example, the subject combination may be used to treat pain or a neurological disorder. Examples of neurological disorders that may be treated with the subject combination include, but are not limited to: affective disorders, psychiatric disorders, cerebral function disorders, movement disorders, dementias, motor neuron diseases, neurodegenerative diseases, seizure disorders, and headaches.

Affective disorders that may be treated by the subject combination include, but are not limited to, depression, major depression, treatment resistant depression, treatment resistant bipolar depression, bipolar disorders including cyclothymia, seasonal affective disorder, mood disorders, chronic depression (dysthymia), psychotic depression, postpartum depression, premenstrual dysphoric disorder (PMDD), situational depression, atypical depression, mania, anxiety disorders, attention deficit disorder (ADD), attention deficit disorder with hyperactivity (ADDH), and attention deficit/hyperactivity disorder (AD/HD), bipolar and manic conditions, obsessive-compulsive disorder, bulimia, obesity or weight-gain, narcolepsy, chronic fatigue syndrome, premenstrual syndrome, substance addiction or abuse, nicotine addiction, psycho sexual dysfunction, pseudobulbar affect, and emotional lability.

Depression may be manifested by depressive symptoms. These symptoms may include psychological changes such as changes in mood, feelings of intense sadness, despair, mental slowing, loss of concentration, pessimistic worry, agitation, anxiety, irritability, guilt, anger, feelings of worthlessness, reckless behavior, suicidal thoughts, or attempts, and/or self-deprecation. Physical symptoms of depression may include insomnia, anorexia, appetite loss, weight loss, weight gain, decreased energy and libido, fatigue, restlessness, aches, pains, headaches, cramps, digestive issues, and/or abnormal hormonal circadian rhythms.

Psychiatric disorders that may be treated by the subject combination, include, but are not limited to, anxiety disorders, including but not limited to, phobias, generalized anxiety disorder, social anxiety disorder, panic disorder, agoraphobia, obsessive-compulsive disorder, and post-traumatic stress disorder (PTSD); mania, manic depressive illness, hypomania, unipolar depression, depression, stress disorders, somatoform disorders, personality disorders, psychosis, schizophrenia, delusional disorder, schizoaffective disorder, schizotypy, aggression, aggression in Alzheimer's disease, agitation, and agitation in Alzheimer's disease.

Agitation in Alzheimer's disease occurs as the disease progresses. Agitation may present itself as inappropriate verbal, emotional, and/or physical behaviors. Inappropriate behaviors may include, but are not limited to, incoherent babbling, inappropriate emotional response, demands for attention, threats, irritability, frustration, screaming, repetitive questions, mood swings, cursing, abusive language, physical outbursts, emotional distress, restlessness, shredding, sleeping disturbances, delusions, hallucinations, pacing, wandering, searching, rummaging, repetitive body motions, hoarding, shadowing, hitting, scratching, biting, combativeness, hyperactivity, and/or kicking.

Alzheimer's disease (AD) is a progressive neurodegenerative disorder characterized by cognitive decline, and behavioral and psychological symptoms including agitation. AD is the most common form of dementia and afflicts an estimated 6 million individuals in the United States, a number that is anticipated to increase to approximately 14 million by 2050. Agitation is reported in up to 70% of patients with AD and is characterized by emotional distress, aggressive behaviors, disruptive irritability, and disinhibition. Managing agitation is a priority in AD. Agitation in patients with AD has been associated with increased caregiver burden, decreased functioning, accelerated cognitive decline, earlier nursing home placement, and increased mortality. There are currently no therapies approved by the FDA for the treatment of agitation in patients with AD.

Cerebral function disorders that may be treated by the subject combination include, but are not limited to, disorders involving intellectual deficits such as senile dementia, Alzheimer's type dementia, memory loss, amnesia/amnestic syndrome, epilepsy, disturbances of consciousness, coma, lowering of attention, speech disorders, voice spasms, Parkinson's disease, Lennox-Gastaut syndrome, autism, hyperkinetic syndrome, and schizophrenia. Cerebral function disorders also include disorders caused by cerebrovascular diseases including, but not limited to, stroke, cerebral infarction, cerebral bleeding, cerebral arteriosclerosis, cerebral venous thrombosis, head injuries, and the like where symptoms include disturbance of consciousness, senile dementia, coma, lowering of attention, and speech disorders.

Substance addiction abuse that may be treated by the subject combination includes, but is not limited to, drug dependence, addiction to cocaine, psychostimulants (e.g., crack, cocaine, speed, meth), nicotine, alcohol, opioids, anxiolytic and hypnotic drugs, cannabis (marijuana), amphetamines, hallucinogens, phencyclidine, volatile solvents, and volatile nitrites. Nicotine addiction includes nicotine addiction of all known forms, such as smoking cigarettes, cigars and/or pipes, e-cigarettes or vaping, and addiction to chewing tobacco.

Movement disorders that may be treated by the subject combination include, but are not limited to, akathisia, akinesia, associated movements, athetosis, ataxia, ballismus, hemiballismus, bradykinesia, cerebral palsy, chorea, Huntington's disease, Huntington's disease chorea, rheumatic chorea, Sydenham's chorea, dyskinesia, tardive dyskinesia, dystonia, blepharospasm, spasmodic torticollis, dopamine-responsive dystonia, Parkinson's disease, restless legs syndrome (RLS), tremor, essential tremor, and Tourette's syndrome, and Wilson's disease.

Dementias that may be treated by the subject combination include, but are not limited to, Alzheimer's disease, Parkinson's disease, vascular dementia, dementia with Lewy bodies, mixed dementia, fronto-temporal dementia, Creutzfeldt-Jakob disease, normal pressure hydrocephalus, Huntington's disease, Wernicke-Korsakoff Syndrome, and Pick's disease.

Motor neuron diseases that may be treated by the subject combination include, but are not limited to, amyotrophic lateral sclerosis (ALS), progressive bulbar palsy, primary lateral sclerosis (PLS), progressive muscular atrophy, post-polio syndrome (PPS), spinal muscular atrophy (SMA), spinal motor atrophies, Tay-Sach's disease, Sandhoff disease, and hereditary spastic paraplegia.

Neurodegenerative diseases that may be treated the subject combination include, but are not limited to, Alzheimer's disease, prion-related diseases, cerebellar ataxia, spinocerebellar ataxia (SCA), spinal muscular atrophy (SMA), bulbar muscular atrophy, Friedrich's ataxia, Huntington's disease, Lewy body disease, Parkinson's disease, amyotrophic lateral sclerosis (ALS or Lou Gehrig's disease), multiple sclerosis (MS), multiple system atrophy, Shy-Drager syndrome, corticobasal degeneration, progressive supranuclear palsy, Wilson's disease, Menkes disease, adrenoleukodystrophy, cerebral autosomal dominant arteriopathy with subcortical infarcts and leukoencephalopathy (CADASIL), muscular dystrophies, Charcot-Marie-Tooth disease (CMT), familial spastic paraparesis, neurofibromatosis, olivopontine cerebellar atrophy or degeneration, striatonigral degeneration, Guillain-Barre syndrome, and spastic paraplegia.

Seizure disorders that may be treated by the subject combination include, but are not limited to, epileptic seizures, nonepileptic seizures, epilepsy, febrile seizures; partial seizures including, but not limited to, simple partial seizures, Jacksonian seizures, complex partial seizures, and epilepsia partialis continua; generalized seizures including, but not limited to, generalized tonic-clonic seizures, absence seizures, atonic seizures, myoclonic seizures, juvenile myoclonic seizures, and infantile spasms; and status epilepticus.

Types of headaches that may be treated by the subject combination include, but are not limited to, migraine, tension, and cluster headaches.

Other neurological disorders that may be treated by the subject combination include, Rett Syndrome, autism, tinnitus, disturbances of consciousness disorders, sexual dysfunction, intractable coughing, narcolepsy, cataplexy; voice disorders due to uncontrolled laryngeal muscle spasms, including, but not limited to, abductor spasmodic dysphonia, adductor spasmodic dysphonia, muscular tension dysphonia, and vocal tremor; diabetic neuropathy, chemotherapy-induced neurotoxicity, such as methotrexate neurotoxicity; incontinence including, but not limited, stress urinary incontinence, urge urinary incontinence, and fecal incontinence; and erectile dysfunction.

In some embodiments, the subject combination may be used to treat pain, joint pain, pain associated with sickle cell disease, pseudobulbar affect, depression (including treatment resistant depression), disorders related to memory and cognition, schizophrenia, Parkinson's disease, amyotrophic lateral sclerosis (ALS), Rhett's syndrome, seizures, cough (including chronic cough), etc.

In some embodiments, the subject combination may be administered orally to relieve musculoskeletal pain including low back pain, and pain associated with rheumatoid arthritis, juvenile rheumatoid arthritis, osteoarthritis, erosive osteoarthritis, sero-negative (non-rheumatoid) arthropathies, non-articular rheumatism, peri-articular disorders, axial spondyloarthritis including ankylosing spondylitis, Paget's disease, fibrous dysplasia, SAPHO syndrome, transient osteoarthritis of the hip, vertebral crush fractures, osteoporosis, etc.

In some embodiments, the subject combination may be administered to relieve inflammatory pain including musculoskeletal pain, arthritis pain, and complex regional pain syndrome.

Arthritis refers to inflammatory joint diseases that can be associated with pain. Examples of arthritis pain include pain associated with osteoarthritis, erosive osteoarthritis, rheumatoid arthritis, juvenile rheumatoid arthritis, sero-negative (non-rheumatoid) arthropathies, non-articular rheumatism, peri-articular disorders, neuropathic arthropathies including Charcot's foot, axial spondyloarthritis including ankylosing spondylitis, and SAPHO syndrome.

In some embodiments, the subject combination is used to treat chronic musculoskeletal pain.

In some embodiments, the subject composition may be administered to relieve complex regional pain syndrome, such as complex regional pain syndrome type I (CRPS-I), complex regional pain syndrome type II (CRPS-II), CRPS-NOS, or another type of CRPS. CRPS is a type of inflammatory pain. CRPS can also have a neuropathic component. Complex regional pain syndrome is a debilitating pain syndrome. It is characterized by severe pain in a limb that can be accompanied by edema, and autonomic, motor, and sensory changes.

In some embodiments, the subject composition may be administered orally to relieve neuropathic pain.

Examples of neuropathic pain include diabetic peripheral neuropathy, post-herpetic neuralgia, trigeminal neuralgia, monoradiculopathies, phantom limb pain, central pain, etc. Other causes of neuropathic pain include cancer-related pain, lumbar nerve root compression, spinal cord injury, post-stroke pain, central multiple sclerosis pain, HIV-associated neuropathy, and radio- or chemo-therapy associated neuropathy, etc.

In some embodiments, the subject composition may be administered to relieve fibromyalgia.

The term “treating” or “treatment” includes the diagnosis, cure, mitigation, treatment, or prevention of disease in man or other animals, or any activity that otherwise affects the structure or any function of the body of man or other animals.

A subject combination may use to treat any disease or condition identified as treatable by the combination of bupropion and dextromethorphan in any of the following U.S. Pat. Nos. 8,569,328, 9,168,234, 9,189,905 9,205,083, 9,238,032, 9,278,095, 9,314,462, 9,370,513, 9,375,429, 9,408,815, 9,421,176, 9,457,023, 9,457,025, 9,474,731, 9,486,450, 9,700,528, 9,700,553, 9,707,191, 9,763,932, 9,861,595, 9,867,819, 9,968,568, 10,058,518, 10,064,857, 10,080,727, 10,092,560, 10,092,561, 10,105,327, 10,105,361, 10,251,879, 10,463,634, 10,512,643, 10,548,857, 10,596,167, 10,772,850, 10,780,064, 10,780,066, 10,786,469, 10,786,496, 10,799,497, 10,806,710, 10,864,209, 10,874,663, 10,874,664, 10,874,665, 10,881,624, 10,881,657, 10,894,046, 10,894,047, 10,898,453, all of which are incorporated by reference herein in their entireties for their disclosure of diseases that may be treated by a combination of bupropion and dextromethorphan, including specific embodiments and combinations described therein.

### Example 1

A Phase 3, randomized, double-blind, active controlled trial was conducted to assess the efficacy and safety of DM/BU in the treatment of treatment resistant depression (TRD). Patients with major depressive disorder (MDD) who had previously failed one or two antidepressant treatments were treated in an open-label fashion with 150 mg bupropion twice daily (300 mg total daily dose) (n=799, n represents of number of patients) during a 6-week lead-in period. Patients who failed to respond to bupropion during this lead-in period were randomized in a 1:1 ratio to treatment with bupropion at this same total daily dose (n=156), or to treatment with DM/BU (45 mg dextromethorphan/105 mg bupropion) twice daily (90 mg dextromethorphan/210 mg bupropion total daily dose) (n=156), for 6 weeks. Inclusion criteria for the open-label period included males or females 18-65 years of age, a history of inadequate response to 1 or 2 prior antidepressant treatments, established by the Antidepressant Treatment Response Questionnaire (ATRQ), and a Hamilton Depression Rating Scale (HAMD-17) total score of ≥18. Inclusion criteria for the double-blind period included inadequate response to 2 or 3 prior antidepressant treatments, including open-label period failure. Exclusion criteria included a history of electroconvulsive therapy, vagus nerve stimulation, transcranial magnetic stimulation or any experimental central nervous system treatment during the current episode or in the past 6 months, schizophrenia, bipolar disorder, obsessive compulsive disorder, psychiatric symptoms secondary to any other general medical condition.

Demographics and baseline characteristics are shown in Table 7 below. Study completion rates were similar across both treatment groups, 89% for dextromethorphan/bupropion and 94% for bupropion.

TABLE 1
Demographics and Baseline Characteristics

| | Dextromethorphan(45 | Bupropion |
|---|---|---|
| | mg)/Bupropion (105 mg) | (150 mg) |
| Age (years) | 44.3 (12.19) | 45.1 (12.56) |
| Female Gender, n (%) | 101 (65.6%) | 97 (62.6%) |
| Race, n (%) | | |
| White | 100 (64.9%) | 106 (68.4%) |
| Black or African American | 41 (26.6%) | 39 (25.2%) |
| Asian | 2 (1.3%) | 6 (3.9%) |
| Other or Not Reported | 11 (7.1%) | 4 (2.6%) |
| BMI (mg/kg²) | 29.9 (5.85) | 29.5 (5.64) |
| MADRS Total Score | 33.4 (5.61) | 33.2 (5.17) |
| CGI-S Score | 4.6 (0.61) | 4.6 (0.54) |

Data are mean (SD) unless otherwise stated
Abbreviations:
BMI = Body Mass Index;
CGI-S = Clinical Global Impression-Severity;
MADRS = Montgomery-Åsberg Depression Rating Scale

The change in depressive symptoms over time was measured using the Montgomery-Åsberg Depression Rating Scale (MADRS) and the Quick Inventory of Depressive Symptomatology-Self-Rated (QIDS-SR-16). The primary endpoint was the change from baseline in the MADRS after 6 weeks of treatment. The key secondary endpoints were the change from baseline in the MADRS after 1 week of treatment, after 2 weeks of treatment, the average change over entire 6-week double-blind treatment period, and the Sheehan Disability Scale (SDS). Other pre-specified secondary efficacy variables included the Cognitive subscale of the Massachusetts General Hospital Cognitive and Physical Functioning Questionnaire (CPFQ), and the Hamilton Anxiety Scale (HAM-A).

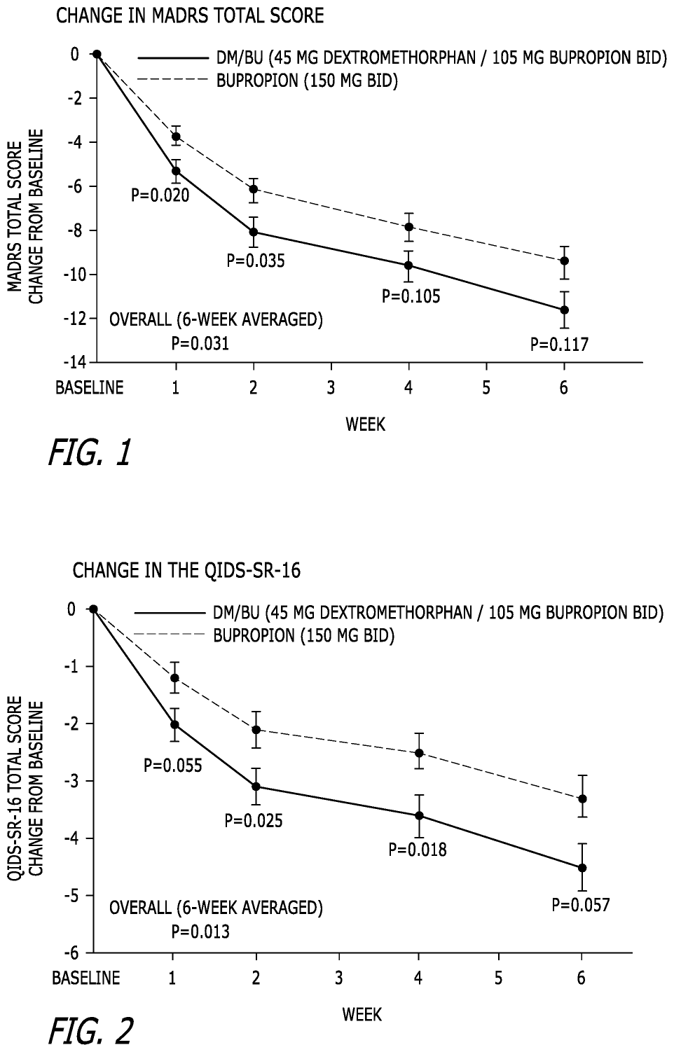

As shown in FIG. 1, DM/BU rapidly and significantly improved symptoms in patients with TRD as measured by MADRS averaged over the entire 6-week treatment period, a key secondary endpoint, with mean reductions of 8.6 for DM/BU (n=154) versus 6.7 for bupropion (n=155) (p=0.031). The rapid onset of action with DM/BU treatment was demonstrated with statistically significant mean MADRS reductions at Week 1, the earliest time point measured, of 5.2 versus 3.6 respectively for DM/BU and bupropion (p=0.02), and at Week 2 of 8.0 versus 6.1 respectively for DM/BU and bupropion (p=0.035), both time points being key secondary endpoints. At Week 6 (primary endpoint), DM/BU demonstrated a numerically greater improvement in MADRS, with mean reductions of 11.6 for DM/BU versus 9.4 for bupropion (p=0.117), but did not reach statistical significance on the Week 6.

As shown in FIG. 2, DM/BU rapidly and significantly improved depressive symptoms in patients with TRD as measured by the Quick Inventory of Depressive Symptomatology-Self-Rated (QIDS-SR-16) averaged over the entire 6-week treatment period, with mean reductions of 3.3 for DM/BU versus 2.3 for bupropion (p=0.013).

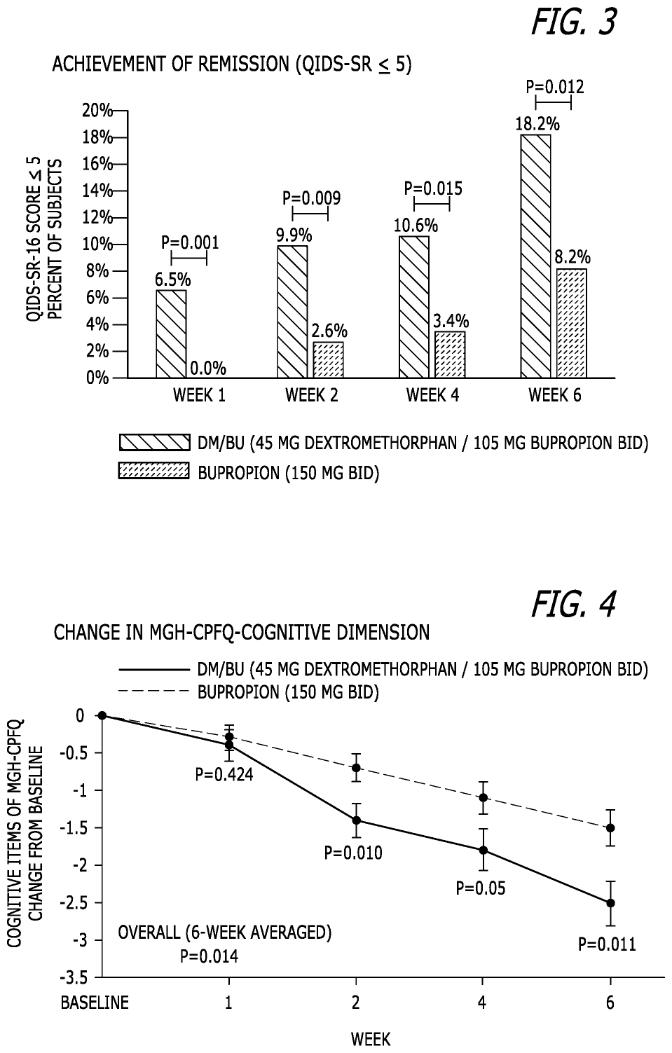

As shown in FIG. 3, rates of remission from depression (defined as QIDS-SR-16≤5) were statistically significantly greater for DM/BU compared to bupropion at Week 1 (p=0.001) and at every time point evaluated thereafter, being achieved by 18.2% of DM/BU patients compared to 8.2% of bupropion patients at Week 6 (p=0.012).

As shown in FIG. 4, DM/BU significantly improved cognitive function in patients with TRD as compared to bupropion, assessed using the Cognitive subscale of the Massachusetts General Hospital Cognitive and Physical Functioning Questionnaire (CPFQ) (p=0.011). Cognitive dysfunction is well documented in the different phases of major depression, and plays an important role in functional recovery from major depression. The improvement in cognitive function with DM/BU was rapid as compared to bupropion, reaching statistical significance as early as Week 2 (p=0.01) and at every time point evaluated thereafter. The Cognitive subscale of the CPFQ assesses sharpness/mental acuity, and the ability to focus/maintain attention, to remember/recall information, and to find words. Statistical significance for the superiority of DM/BU versus bupropion was also achieved for the entire CPFQ (p=0.014), which assesses physical in addition to cognitive functioning.

DM/BU rapidly and significantly reduced anxiety symptoms in patients with TRD as compared to bupropion, assessed using the Hamilton Anxiety Scale (HAM-A) (p=0.009). DM/BU demonstrated numerical improvement versus the active comparator bupropion for all other efficacy variables assessed.

DM/BU was well tolerated in the trial. The most commonly reported adverse events in the DM/BU arm were dizziness and nausea. The rates of discontinuation due to adverse events were low in both treatment groups (2.6% for DM/BU and 1.9% for bupropion). There were three serious adverse events in the DM/BU arm, consisting of migraine; overdose; and suicidal ideation, which occurred more than one week after the cessation of treatment. Treatment with DM/BU was not associated with psychotomimetic effects, weight gain, or sexual dysfunction. Adverse events are listed in Table 8 below.

TABLE 2
Treatment-Emergent Adverse Events

| | Double-blind Period^{b} | | Open-label Period |
|---|---|---|---|
| | DM/BU | BU | BU |
| | (N = 154) | (N = 156) | (n = 310) |
| Any TEAE^{a} | 67 (43.5%) | 61 (39.1%) | 135 (43.5) |
| Dizziness | 13 (8.4%) | 0 | 9 (2.9%) |
| Nausea | 8 (5.2%) | 3 (1.9%) | 22 (7.1%) |
| Dry mouth | 6 (3.9%) | 3 (1.9%) | 13 (4.2%) |
| Headache | 4 (2.6%) | 7 (4.5%) | 14 (4.5%) |
| Insomnia | 3 (1.9%) | 5 (3.2%) | 19 (6.1%) |
| Constipation | 3 (1.9%) | 3 (1.9%) | 13 (4.2%) |
| Anxiety | 2 (1.3%) | 0 | 11 (3.5%) |
| Irritability | 0 | 2 (1.3%) | 10 (3.2%) |

Abbreviations:
AE = adverse event.
Data presented as number of subjects (% of subjects)
^{a}Treatment-emergent AEs occurring in ≥3 subjects during the open-label period or ≥5% of subjects during the double-blind period are reported.
^{b}In double-blind period, treatment-emergent AE is defined as any AE with an onset on or after date of randomization and prior to or on visit 9 date or period 2 early termination date.

### Example 2

From the clinical trial in Example 1, a study was carried out to evaluate the efficacy of the combination of dextromethorphan and bupropion in patients who were treatment-adherent, based on an analysis of plasma drug concentrations. It was discovered in this study that measurement of plasma drug concentrations is a direct method of assessing treatment adherence. Treatment adherence may improve the outcome with the use of a combination of dextromethorphan and bupropion since the attainment of therapeutic concentrations may be predicated not only on dose and frequency of the study drug but also on the time-dependent inhibition of CYP2D6.

In the patients of Example 1, plasma samples were collected after up to 6 weeks of treatment in the clinical trial (end of treatment visit) and analyzed. Treatment adherence was assessed based on the following plasma drug concentration cutoffs:
DM/BU arm: dextromethorphan concentration ≥15 ng/mL
Bupropion SR arm: bupropion concentration ≥10 ng/mL

The selected drug concentration cutoffs approximate the lowest observed concentrations 24 hours after the last dose of the study drug under 100% adherent conditions. The 15 ng/mL threshold at 24 hours after the last dose corresponds to a plasma concentration of about 19 ng/mL at about 12 hours after a dose that is administered after steady state has been achieved (e.g., day 42).

The primary endpoint was the change from baseline in the MADRS after 6 weeks of treatment. The key secondary endpoints were the change from baseline in the MADRS after 1 week and 2 weeks of treatment, the overall change in the MADRS total score, and change from baseline in the Sheehan Disability Scale (SDS) at week 6. Other efficacy assessments included Quick Inventory Depressive Symptoms-Self Report (QIDS-SR-16), Hamilton Anxiety Rating Scale, and Cognition and Physical Functioning Questionnaire (CPFQ).

The demographics and baseline characteristics of the treatment adherent patients are shown in Table 3 below.

TABLE 3
Demographics and Baseline Characteristics

| | Dextromethorphan (45 | Bupropion |
|---|---|---|
| | mg)/Bupropion (105 mg) | (150 mg) |
| Age (years) | 44.4 (12.75) | 45.7 (12.98) |
| Female Gender, n (%) | 82 (65.6%) | 82 (62.6%) |
| Race, n (%) | | |
| White | 82 (67.2%) | 94 (71.8%) |
| Black or African American | 28 (23.0%) | 28 (21.4%) |
| Asian | 2 (1.6%) | 5 (3.8%) |
| BMI (mg/kg²) | 29.8 (5.92) | 29.6 (5.66) |
| MADRS Total Score | 33.6 (5.44) | 33.0 (5.11) |
| CGI-S Score | 4.6 (0.61) | 4.6 (0.54) |

Data are mean (SD) unless otherwise stated
Abbreviations:
BMI = Body Mass Index;
CGI-S = Clinical Global Impression-Severity;
MADRS = Montgomery-Åsberg Depression Rating Scale

Demographics and baseline characteristics were similar across both treatment groups. Efficacy analysis based on subjects who were treatment-adherent based on the following plasma concentrations:
DM/BU treated subjects: dextromethorphan concentration of ≥15 ng/mL (N=122)
Bupropion treated subjects: bupropion concentration of ≥10 ng/mL (N=131)

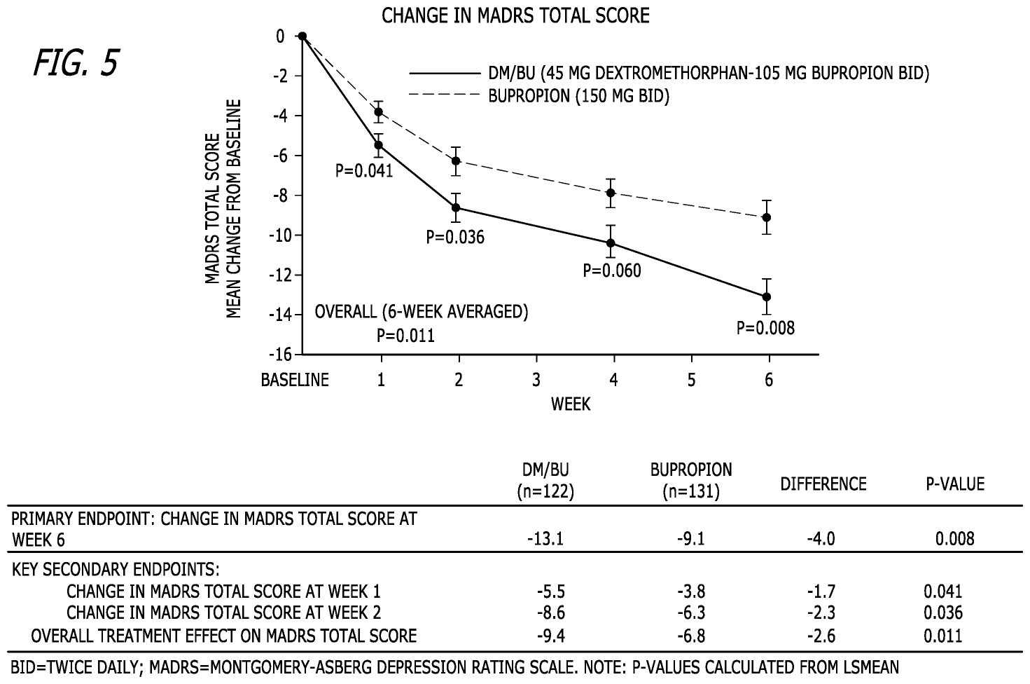

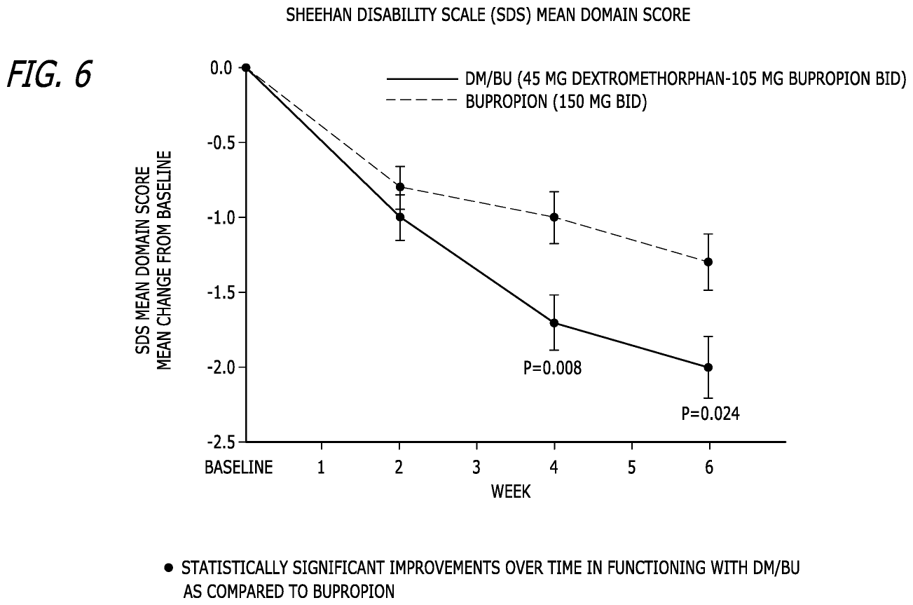

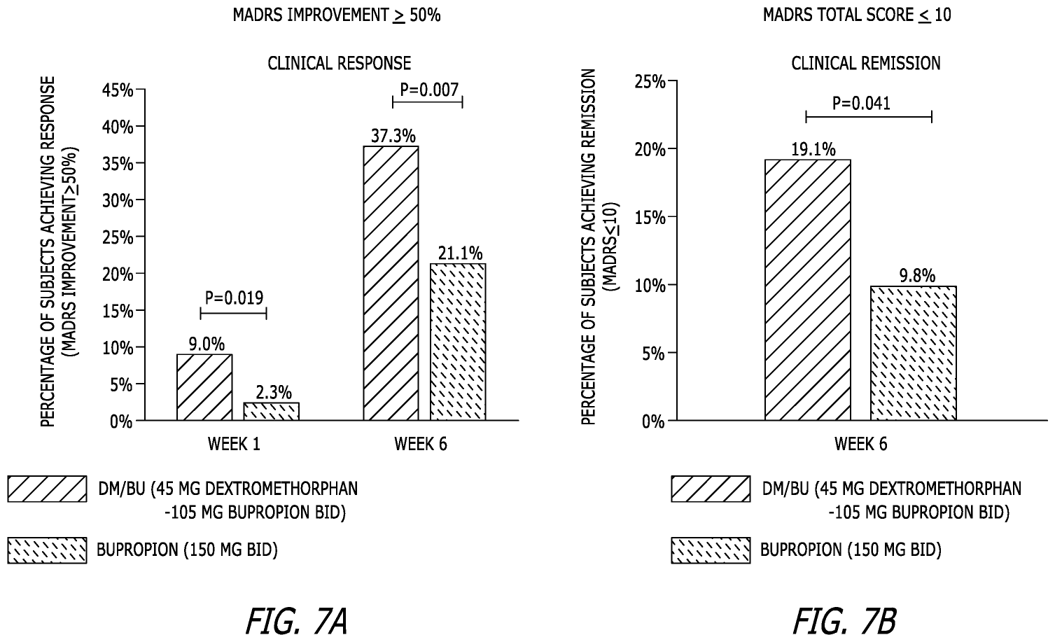

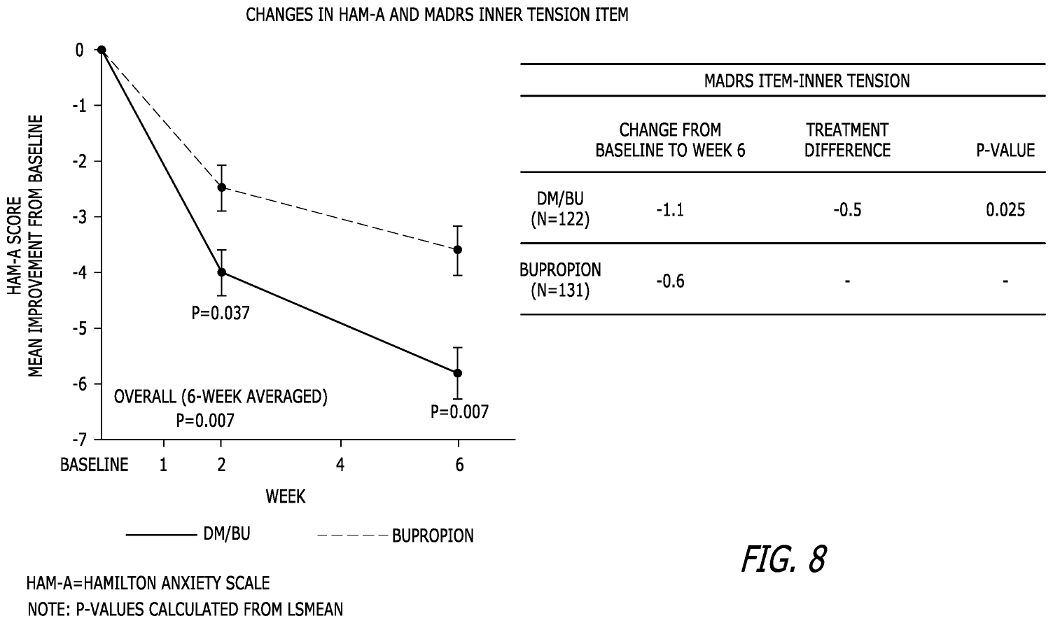

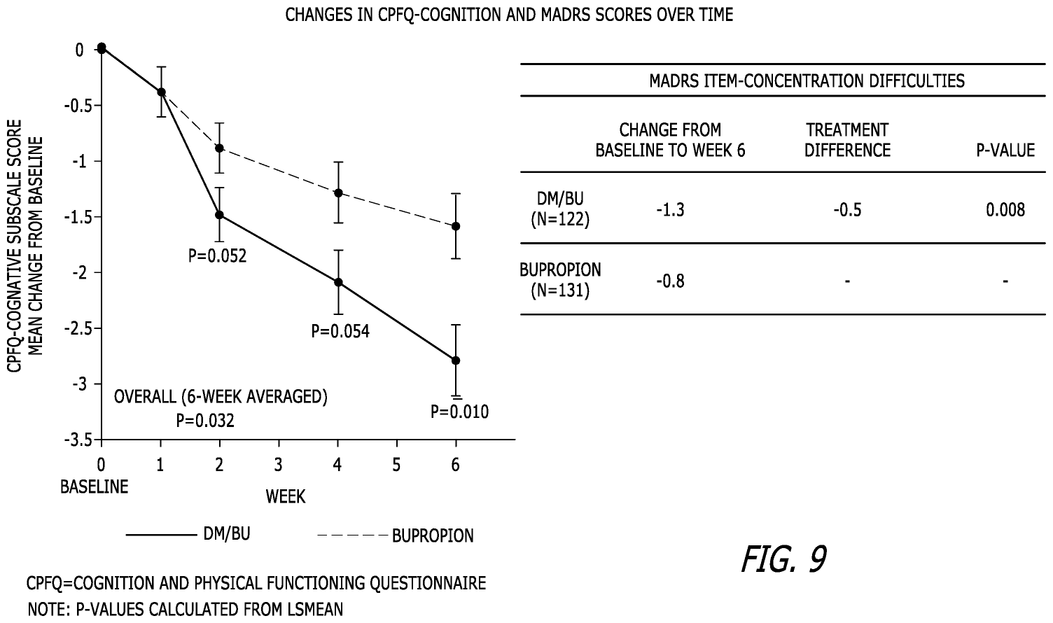

82% of subjects were adherent to study medication based on plasma concentrations. DM/BU rapidly and substantially improved depressive symptoms in treatment-adherent patients with treatment-resistant depression. DM/BU demonstrated statistically significant improvements from baseline in the MADRS total score, as compared to bupropion, at all timepoints, including at Week 1, the earliest timepoint assessed (FIG. 5). Treatment with DM/BU was associated with statistically significant improvements in functioning on the SDS, as compared to bupropion (FIG. 6). Statistically significantly greater rates of clinical remission and response were achieved with DM/BU as compared to bupropion (FIG. 7A and FIG. 7B). BU/DM also demonstrated significant improvements in anxiety (FIG. 8) and cognition (FIG. 9) compared to bupropion.

DM/BU was generally safe and well tolerated in the trial (Table 4).

TABLE 4
Treatment-Emergent Adverse Events

| | BU/DM | Bupropion |
|---|---|---|
| | (N = 122) | (N = 132) |
| Dizziness | 12 (9.8%) | 0 (0.0%) |
| Nausea | 6 (4.9%) | 2 (1.5%) |
| Dry mouth | 6 (4.9%) | 3 (2.3%) |
| Headache | 4 (3.3%) | 6 (4.5%) |
| Somnolence | 5 (4.1%) | 2 (1.5%) |
| Nasopharyngitis | 4 (3.3%) | 0 (0.0%) |
| Blood pressure increased | 4 (3.3%) | 0 (0.0%) |

Rates of discontinuation due to adverse events were low in both groups; 2.5% for DM/BU and 0.8% for bupropion.

1. A method of treating major depressive disorder in a human patient in need thereof, comprising administering a combination comprising about 45 mg of dextromethorphan hydrobromide, or a molar equivalent amount of another salt form or the free base form of dextromethorphan, and about 105 mg of bupropion hydrochloride, or a molar equivalent amount of another salt form or the free base form of bupropion, twice a day for at least six weeks to the human patient, wherein, twelve hours after a dose of the combination is administered on day 42, the human patient has a plasma level of dextromethorphan that is at least about 19 ng/mL.

2. A method of treating major depressive disorder a human patient in need thereof, comprising administering a dosage form comprising about 45 mg of dextromethorphan hydrobromide, or a molar equivalent amount of another salt form or the free base form of dextromethorphan, and about 105 mg of bupropion hydrochloride, or a molar equivalent amount of another salt form or the free base form of bupropion, twice a day for at least six weeks to the human patient, wherein twenty-four hours after administration of the dosage form is discontinued, the human patient has a plasma level of dextromethorphan that is at least about 15 ng/mL.

3. The method of claim 1, wherein the major depressive disorder is treatment-resistant depression.

4. The method of claim 1, wherein the combination is in a tablet which is orally administered once daily to the human patient for three days, and after three days, the tablet is orally administered twice daily, wherein the tablet contains about 105 mg of bupropion hydrochloride and about 45 mg of dextromethorphan hydrobromide.

5. The method of claim 1, wherein the dextromethorphan hydrobromide is in an immediate-release formulation.

6. The method of claim 1, wherein the bupropion hydrochloride is in an extended-release formulation.

7. The method of claim 4, wherein the tablet is a bilayer tablet.

8. The method of claim 1, wherein the Tₘₐₓ of dextromethorphan is about 3 hours.

9. The method of claim 1, wherein, at steady state plasma levels of dextromethorphan, the accumulation ratio of dextromethorphan is about 20 based on the Cₘₐₓ.

10. The method of claim 1, wherein, at steady state plasma levels of dextromethorphan, the accumulation ratio of dextromethorphan is about 32 based on the AUC₀₋₁₂.

11. The method of claim 2, wherein the major depressive disorder is treatment-resistant depression.

12. The method of claim 2, wherein the combination is in a tablet which is orally administered once daily to the human patient for three days, and after three days, the tablet is orally administered twice daily, wherein the tablet contains about 105 mg of bupropion hydrochloride and about 45 mg of dextromethorphan hydrobromide.

13. The method of claim 2, wherein the dextromethorphan hydrobromide is in an immediate-release formulation.

14. The method of claim 2, wherein the bupropion hydrochloride is in an extended-release formulation.

15. The method of claim 12, wherein the tablet is a bilayer tablet.

16. The method of claim 2, wherein the Tₘₐₓ of dextromethorphan is about 3 hours.

17. The method of claim 2, wherein, at steady state plasma levels of dextromethorphan, the accumulation ratio of dextromethorphan is about 20 based on the Cₘₐₓ.

18. The method of claim 2, wherein, at steady state plasma levels of dextromethorphan, the accumulation ratio of dextromethorphan is about 32 based on the AUC₀₋₁₂.
